(19) United States
(12) Patent Application Publication
(10) Pub. No.: US 2009/0005687 A1
(43) Pub. Date: Jan. 1, 2009

(54) ULTRASONIC IMAGING APPARATUS

(76) Inventor: Sotaro Kawae, Tokyo (JP)

Correspondence Address: Patrick W. Rasche;Armstrong Teasdale LLP, Suite 2600, One Metropolitan Square, St. Louis, MO, 63102, US

(21) Appl. No.: 12/146,316

(22) Filed: Jun. 25, 2008

(30) Foreign Application Priority Data
Jun. 27, 2007 (JP) 2007-168475

(51) Int. Cl. A61B 8/00

(52) U.S. Cl. 600/461

(57) ABSTRACT

An ultrasonic imaging apparatus includes an ultrasonic probe which captures 3D tomographic image data, a puncture needle which is attached to a puncture guide and introduced into a 3D region of a subject on which the 3D tomographic image data is captured, and an image processor which forms image data on an introduction sectional plane including an expected introduction path along which the introduction is expected. The image processor includes a monitor area setting device which sets, in a 3D memory area, a monitor memory area which corresponds to a planar monitor area in the 3D region which the expected introduction path penetrates, a penetration point detecting device which detects a point of penetration of the monitor area by the puncture needle according to tomographic image data, and a sectional plane position correcting device which corrects the position of the introduction sectional plane to include the penetration point.

### CROSS REFERENCE TO RELATED APPLICATIONS

This application claims the benefit of Japanese Patent Application No. 2007-168475 filed Jun. 27, 2007, which is hereby incorporated by reference in its entirety.

### BACKGROUND OF THE INVENTION

The subject matter disclosed herein relates to an ultrasonic imaging apparatus that makes a puncture while capturing tomographic image data on a subject.

In recent years, ultrasonic imaging apparatuses have been used to carry out a biopsy while monitoring a puncture needle introduced into a subject. By doing so, a puncture is properly made in the target affected site of the subject to take a sample of the subject properly. Here, the puncture needle is introduced along a puncture guide held by an ultransonic probe. Consequently the introduction path of the puncture needle runs along the sectional plane of the ultransonic probe and the puncture needle introduced from a prescribed point and a prescribed direction in the sectional plane is observed using an ultransoic imaging apparatus (for example, see Japanese Patent Application No. 2003-334191).

Regarding ultrasonic probes of ultrasonic imaging apparatuses, those which capture 2D tomographic image data on a given sectional plane and at the same time those which capture 3D tomographic image data on a 3D imaging region have been increasingly used. Among ultrasonic probes that capture 3D tomographic image data are those that have a 2D array arrangement of piezoelectric elements on a plane of contact with a subject and those that have a one-dimensional array arrangement of piezoelectric elements for mechanical scanning.

Even when these ultrasonic probes are used, biopsy using a puncture needle as mentioned above is carried out similarly and the puncture needle is introduced using a puncture guide along an electronic scan direction in which piezoelectric elements are arranged in an array pattern.

However, according to the above background art, the introduction path of the puncture needle may not fall within a sectional plane and may be hard to observe. In other words, the introduction path of the puncture needle may not be an expected introduction path as defined by the puncture guide because of the following reasons: the outer diameter of the puncture needle is small (1 mm or so), the inside of a subject is resilient and includes tissues with different strengths, and the puncture guide has some irregularity.

Also, in an ultrasonic probe with a 3D imaging range, a sectional plane is displayed as a result of extracting tomographic image data on a sectional plane including an expected introduction path from captured 3D tomographic image data. By moving this sectional plane successively, a sectional plane including the introduction path of the puncture needle can be obtained; however, it is troublesome and inefficient for the operator who is introducing the needle.

For the above reasons, it is important to realize an ultrasonic imaging apparatus that efficiently finds a sectional plane including the introduction path of the puncture needle and properly makes an image of the sectional plane including the introduction path.

### BRIEF DESCRIPTION OF THE INVENTION

It is desirable that the problems described previously are solved.

An ultrasonic imaging apparatus according to a first aspect of the invention includes an ultrasonic probe which captures 3D tomographic image data on a subject, a puncture guide which is held by the ultrasonic probe, a puncture needle which is attached to the puncture guide and introduced into a 3D region of a subject on which the 3D tomographic image data is captured, an image memory which stores the 3D tomographic image data, and an image processor which forms, according to the 3D tomographic image data, image data on an introduction sectional plane including an expected introduction path along which the introduction is expected to be made, wherein the image processor comprises a monitor area setting device which sets, in a 3D memory area of the image memory corresponding to the 3D region, a monitor memory area which corresponds to a planar monitor area in the 3D region which the expected introduction path penetrates in the vicinity of the center, a penetration point detecting device which detects a point of penetration of the monitor area by the puncture needle according to tomographic image data in the monitor memory area when the puncture needle penetrates the monitor area, and a sectional plane position correcting device which corrects the position of the introduction sectional plane so as to include the penetration point.

According to the first aspect of the invention, in the image processor, the monitor area setting device sets, in a 3D memory area of the image memory corresponding to the 3D region, a monitor memory area which corresponds to a planar monitor area in the 3D region which the expected introduction path penetrates in the vicinity of the center, the penetration point detecting device detects a point of penetration of the monitor area by the puncture needle according to tomographic image data in the monitor memory area when the puncture needle penetrates the monitor area, and the sectional plane position correcting device corrects the position of the introduction sectional plane so as to include the penetration point.

The ultrasonic imaging apparatus according to a second aspect of the invention is characterized in that in the ultrasonic imaging apparatus according to the first aspect, the ultrasonic probe includes a probe array in which piezoelectric elements are one-dimensionally arranged in an arc or linear pattern, and a mechanical scan device which mechanically scans the probe array repeatedly in a mechanical scan direction orthogonal to the direction of the one-dimensional arrangement.

According to the second aspect of the invention, the ultrasonic probe captures 3D tomographic image data by a mechanical scan.

The ultrasonic imaging apparatus according to a third aspect of the invention is characterized in that in the ultrasonic imaging apparatus according to the second aspect, the puncture guide introduces the puncture needle into a sectional plane including the direction of the arrangement, which is located in the center of the scan area to be mechanically scanned.

According to the third aspect of the invention, the puncture probe is introduced into the center of the plane of contact with the subject.

The ultrasonic imaging apparatus according to a fourth aspect of the invention is characterized in that in the ultrasonic imaging apparatus according to the third aspect, the image processor identifies the sectional plane including the arrangement direction located in the center of the scan area to be mechanically scanned, as the introduction sectional plane.

According to the fourth aspect of the invention, the image processor forms tomographic image data on the sectional plane into which the puncture needle is introduced.

The ultrasonic imaging apparatus according to a fifth aspect of the invention is characterized in that in the ultrasonic imaging apparatus according to the first aspect, the ultrasonic probe includes a 2D probe array in which piezoelectric elements are two-dimensionally arranged in a planar pattern.

According to the fifth aspect of the invention, 3D tomographic image data is captured by an electronic scan.

The ultrasonic imaging apparatus according to a sixth aspect of the invention is characterized in that in the ultrasonic imaging apparatus according to any of the first to fifth aspects, the image processor includes orthogonal 3-sectional plane formation device to form tomographic image data on three orthogonal sectional planes, including the introduction sectional plane as one sectional plane, which are orthogonal to each other in the 3D region.

The ultrasonic imaging apparatus according to a seventh aspect of the invention is characterized in that the ultrasonic imaging apparatus according to the sixth aspect further includes a display section which displays tomographic image data on the three orthogonal sectional planes.

According to the seventh aspect of the invention, the condition of the 3D region can be easily grasped from images of the three orthogonal sectional planes.

The ultrasonic imaging apparatus according to an eighth aspect of the invention is characterized in that in the ultrasonic imaging apparatus according to the seventh aspect, the monitor area setting device extracts tomographic image data in the monitor memory area from the image memory and displays tomographic images of the tomographic image data on the display section.

According to the eighth aspect of the invention, an image of the monitor area can be observed.

The ultrasonic imaging apparatus according to a ninth aspect of the invention is characterized in that in the ultrasonic imaging apparatus according to the eighth aspect, the penetration point detecting device includes a cursor position detecting device which detects a cursor for specifying a point in the displayed tomographic image and the position of the cursor in the tomographic image and identifies it as the penetration point.

According to the ninth aspect of the invention, the penetration point of the puncture needle which has penetrated the monitor area is manually detected by an operator.

The ultrasonic imaging apparatus according to a tenth aspect of the invention is characterized in that in the ultrasonic imaging apparatus according to any of the first to eighth aspects, the penetration point detecting device includes an automatic detecting device which extracts tomographic image data in the monitor memory area from the 3D tomographic image data and automatically detects the penetration point of the puncture needle as included in a tomographic image of the tomographic image data.

According to the tenth aspect of the invention, the penetration point of the puncture needle which has penetrated the monitor area is automatically detected without bothering an operator.

The ultrasonic imaging apparatus according to an eleventh aspect of the invention is characterized in that in the ultrasonic imaging apparatus according to the tenth aspect, the automatic detecting device automatically detects the pixel position of a high-brightness spot beyond a threshold in the tomographic image, as the penetration point.

According to the eleventh aspect of the invention, the position of the cutting edge of the puncture needle which forms a high-brightness spot is detected as the penetration point.

The ultrasonic imaging apparatus according to a twelfth aspect of the invention is characterized in that in the ultrasonic imaging apparatus according to the tenth or eleventh aspect, when the plurality of 3D tomographic image data captured temporally successively are stored in the image memory, the automatic detecting device extracts a plurality of temporally successive tomographic image data in the monitor memory area, and automatically detects the penetration point from tomographic images of the plural tomographic image data.

According to the twelfth aspect of the invention, the cutting edge of the puncture needle penetrating the monitor area is properly detected.

The ultrasonic imaging apparatus according to a thirteen aspect of the invention is characterized in that in the ultrasonic imaging apparatus according to the tenth aspect, when the plurality of 3D tomographic image data captured temporally successively are stored in the image memory, the automatic detecting device extracts a plurality of temporally successive 2D tomographic image data in the monitor memory area, differentiates between two 2D tomographic images captured at different times in 2D tomographic images of the plural 2D tomographic image data, and automatically detects the pixel position of a spot beyond a threshold as included in the difference tomographic image obtained by the differentiation, as the penetration point.

According to the thirteenth aspect of the invention, the penetration point is detected according to brightness change that occurs when the puncture needle penetrates the monitor area.

The ultrasonic imaging apparatus according to a fourteenth aspect of the invention is characterized in that in the ultrasonic imaging apparatus according to any of the first to thirteenth aspects, the monitor area setting device includes a first monitor memory area in the image memory corresponding to a first monitor area located in the 3D region's periphery where the puncture needle begins to be introduced into the 3D region.

According to the fourteenth aspect of the invention, the monitor area is set in a position where the puncture needle begins to be introduced so that the introduction path is identified early.

The ultrasonic imaging apparatus according to a fifteenth aspect of the invention is characterized in that in the ultrasonic imaging apparatus according to the fourteenth aspect, the monitor area setting device includes a second monitor memory area in the image memory corresponding to a second monitor area located within the 3D region.

According to the fifteenth aspect of the invention, the position of the puncture needle is identified in the 3D region before the puncture needle reaches the affected site.

The ultrasonic imaging apparatus according to a sixteenth aspect of the invention is characterized in that in the ultrasonic imaging apparatus according to the fifteenth aspect, according to the tomographic image data in the first monitor memory area and the second monitor memory area, the penetration point detecting device detect a first penetration point and a second penetration point where the puncture needle penetrates the first monitor area and the second monitor area respectively.

According to the sixteenth aspect of the invention, the introduction path of the puncture needle is properly identified by two points, the first penetration point and the second penetration point.

The ultrasonic imaging apparatus according to a seventeenth aspect of the invention is characterized in that in the ultrasonic imaging apparatus according to the sixteenth aspect, the sectional plane position correcting device corrects the position of the introduction sectional plane so as to include the first penetration point and the second penetration point.

According to the seventeenth aspect of the invention, the introduction sectional plane surely includes the introduction path.

According to the invention, it is easy to make the introduction sectional plane include the introduction path of the puncture needle and consequently the introduction path can be observed properly and a sample of the subject can be taken properly in biopsy.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the general structure of an ultrasonic imaging apparatus.

FIG. 2 is an explanatory view showing the shape of the cutting edge of a puncture needle according to the first embodiment.

FIG. 3 is a sectional view showing the mechanical structure of an ultrasonic probe according to the first embodiment.

FIG. 4 is an explanatory view showing electronic scan and mechanical scan by the ultrasonic probe according to the first embodiment.

FIG. 5 is a functional block diagram showing the functional structure of the control section of the ultrasonic imaging apparatus according to the first embodiment.

FIG. 6 is an explanatory view showing three orthogonal sectional planes in a 3D region that are imaged by the ultrasonic probe.

FIG. 7 is an explanatory view showing image data on three orthogonal sectional planes displayed on a display section.

FIG. 8 is an explanatory view showing a monitor area in the 3D region according to the first embodiment.

FIG. 9 is a flowchart showing operation of the control section according to the first embodiment.

FIG. 10 is an explanatory view showing an example of a high-brightness spot displayed in a monitor image.

FIG. 11 is an explanatory view showing correction of the position of a sectional plane A according to the first embodiment.

FIG. 12 is an explanatory view showing a first monitor area and a second monitor area according to the second embodiment.

### DETAILED DESCRIPTION OF THE INVENTION

Next, the best mode for carrying out the invention as an ultrasonic imaging apparatus will be described referring to the accompanying drawings. It does not limit the invention.

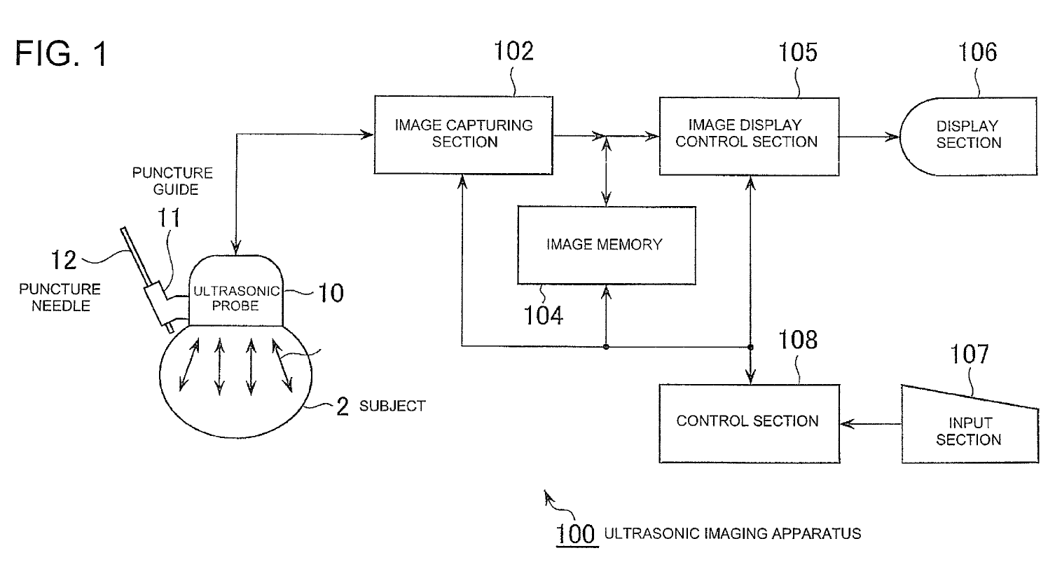

First, the general structure of an ultrasonic imaging apparatus 100 according to the first embodiment will be described. FIG. 1 is a block diagram showing the general structure of the ultrasonic imaging apparatus 100 according to the first embodiment. The ultrasonic imaging apparatus 100 includes an ultrasonic probe 10, a puncture guide 11, a puncture needle 12, an image capturing section 102, an image memory 104, an image display control section 105, a display section 106, an input section 107 and a control section 108.

The ultrasonic probe 10 is a part which transmits or receives ultrasonic waves, namely radiates ultrasonic waves in a specific direction of a sectional plane of a subject 2 and receives ultrasonic echoes reflected each time from the inside of the subject 2 as temporally successive sound rays. On the other hand, the ultrasonic probe 10 performs an electronic scan or mechanical scan while changing the direction of ultrasonic radiation successively. As will be detailed later, the ultrasonic probe 10, which includes a probe array as piezoelectric elements arranged in an array pattern in the electronic scan direction, and a mechanical scanning means for mechanically scanning the probe array orthogonally to this arrangement, captures 3D tomographic image information.

The puncture guide 11, attached to the gripper of the ultrasonic probe 10, introduces the attached puncture needle 12 into a given sectional plane of the subject 2.

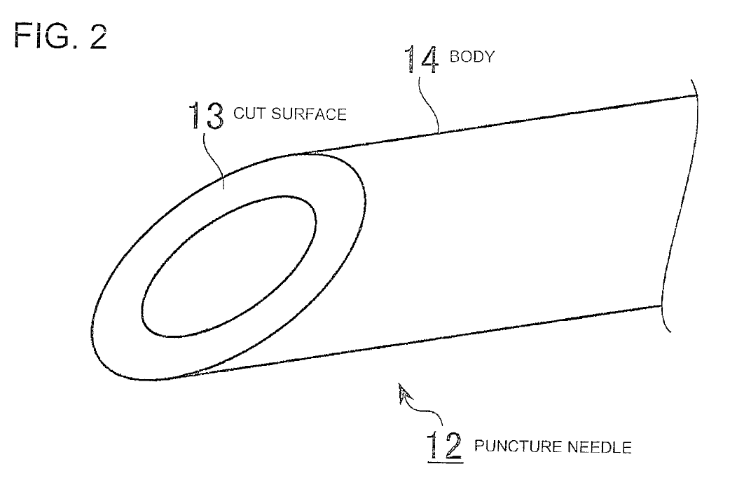

The puncture needle 12 is an injection needle of stainless steel or the like and the outer diameter varies from approximately 0.3 millimeters (mm) to approximately 4.0 mm depending on the application purpose. In a biopsy that takes a sample from the inside of the subject 2, a biopsy needle is placed inside the puncture needle 12 and a sample is taken using this biopsy needle. Generally the cutting edge of the puncture needle 12 has an acute-angled cut surface 13 as shown in FIG. 2. The cut surface 13 of the cutting edge provides a higher reflected ultrasonic echo intensity than the cylindrical body 14 of the puncture needle 12 and thus it is observed as a high-brightness spot in a tomographic image. On the other hand, the body of the puncture needle 12 provides a lower reflected ultrasonic echo intensity than tissues of the subject 2 and thus it is a low-brightness area which is hard to observe.

Again referring to FIG. 1, the image capturing section 102 includes a transducer, a B mode processor, and a Doppler processor. The transducer, which is connected with the ultrasonic probe 10 by a coaxial cable, generates an electrical signal to activate the piezoelectric elements of the ultrasonic probe 10. The transducer also carries out first-stage amplification of reflected ultrasonic echoes that it has received.

The B mode processor performs processing to generate in real time a B mode image from a reflected ultrasonic echo signal amplified by the transducer in real and the Doppler processor extracts phase change information from the reflected ultrasonic echo signal amplified by the transducer and calculates, in real time, blood flow data such as average velocity as average frequency in frequency shift, power and variance.

The image memory 104 is a large capacity memory which stores the B mode image data captured by the image capturing section 102, Doppler image data including blood flow data and similar data. The image memory 104 is, for example, a hard disk or the like.

The image display control section 105 performs display frame rate conversion for the B mode image data generated by the B mode processor and the blood flow data generated by the Doppler processor and controls the image display form or position.

The display section 106 includes a CRT (Cathode Ray Tube) or LCD (Liquid Crystal Display) and displays a B mode image or Doppler image or the like.

The input section 107 includes a keyboard and a mouse or the like and an operator enters an operation input signal through it. At the input section 107, an entry operation is made to select a B mode display or Doppler processing display, an entry operation with a cursor or the like is made to perform image processing of the displayed image data or an entry operation is made to specify the Doppler imaging area for Doppler processing. Furthermore, information for mechanically scanning the probe array of the ultrasonic probe 10 such as scan mode, mechanical scan speed, maximum oscillation angle and scan start is sent from the input section 107 to the control section 108.

The control section 108 includes an image capture controller which controls operation of components of the ultrasonic imaging apparatus including the ultrasonic probe according to the operation input signal entered through the input section 107 and the stored program or data, and an image processor which performs image processing using 3D tomographic image data stored in the image memory 104. The image capture controller controls the position of the probe array inside the ultrasonic probe 10 according to information on the ultrasonic probe 10 sent from the input section 107 such as scan mode, mechanical scan speed, maximum oscillation angle and scan start. The image processor will be described in detail later.

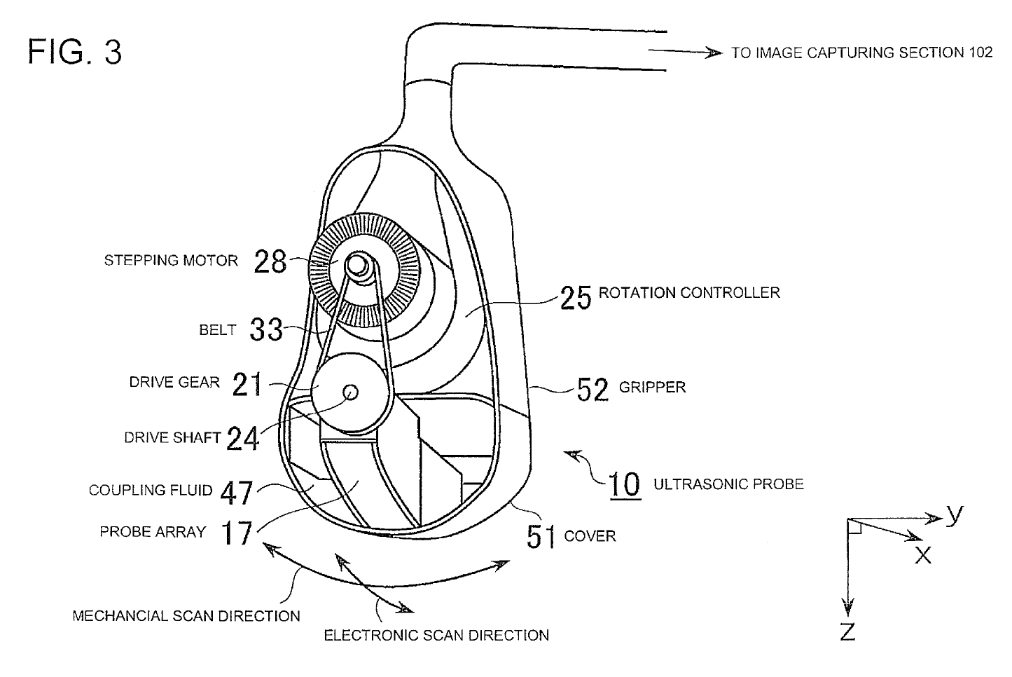

FIG. 3 is a probe sectional view showing the internal structure of the ultrasonic probe 10. The ultrasonic probe 10 includes a cover 51, a gripper 52, a probe array 17, a coupling fluid 47, and a drive gear 21, a drive shaft 24, a stepping motor 28, a belt 33 and a rotation controller 25 which constitute a mechanical scanning means. Here the cover 51 and gripper 52 constitute a case which houses the probe array 17, coupling fluid 47, and the drive gear 21, stepping motor 28, belt 33 and rotation controller 25 which constitute the mechanical scanning means. The x, y and z coordinate axes shown in the figure are common to all the drawings and suggest positional relations among the drawings.

The cover 51, made of translucent film, has the shape of an arc along the trajectory of the probe array 17 to be mechanically scanned in an arc pattern. The material of the cover 51 is an acoustic impedance material that transmits ultrasonic waves generated by the probe array 17 and reflected ultrasonic echoes from the subject 2 with low loss.

The gripper 52 is made of moldable plastic or similar material and has a shape that allows the operator to hold the ultrasonic probe 10 easily and firmly. The puncture guide 11 can be attached to, or detached from, the gripper 52.

The probe array 17 has a convex linear scan type probe. This linear scan type probe has a plurality of piezoelectric elements arranged in an array pattern in the electronic scan direction orthogonal to the mechanical scan direction and performs an electronic scan along this arrangement.

The probe array 17 performs a mechanical scan in the mechanical scan direction by the mechanical scanning means. The mechanical scanning means has the drive shaft 24, an oscillating means for a turn toward the electronic scan direction and as the drive shaft 24 rotates, the probe array 17's probe surface in contact with the cover 51 oscillates along an arc trajectory in the mechanical scan direction. The inside of the cover 51 in which the probe array 17 lies is filled with coupling fluid 47 where there is least loss in acoustic coupling between the probe array 17 and the cover 51.

The drive shaft 24 is mechanically connected with the stepping motor 28 through the drive gear 21 and belt 33. The stepping motor 28 rotates a given angle with high accuracy as intended upon input of control pulses from the rotation controller 25. This rotation rotates the mechanically connected drive shaft 24 and probe array 17 in the mechanical scan direction.

The rotation controller 25 has a pulse generator that generates pulses to drive the stepping motor 28 and a pulse controller which controls the pulses. The rotation controller 25 controls the rotation angle for the stepping motor 28 and probe array 17 according to control information from the image capturing section 10 to oscillate the probe array 17 around the drive shaft 24 as the center of rotation.

For example, when no scan is made, the rotation controller 25 lets the probe array 17 stay at the home position where it is oriented in the z-axis direction fully facing the subject. Then, according to the probe array 17's maximum oscillation angle measured from its frontal position opposite to the subject and the probe array 17's scan speed in the mechanical scan direction which have been entered through the input section 107 by an operator, the rotation controller 25 starts scanning from the home position in a given mechanical scan direction. After that, upon receipt of an instruction to stop scanning from the input section 107, the rotation controller 25 returns the probe array 17 to the home position and stops scanning.

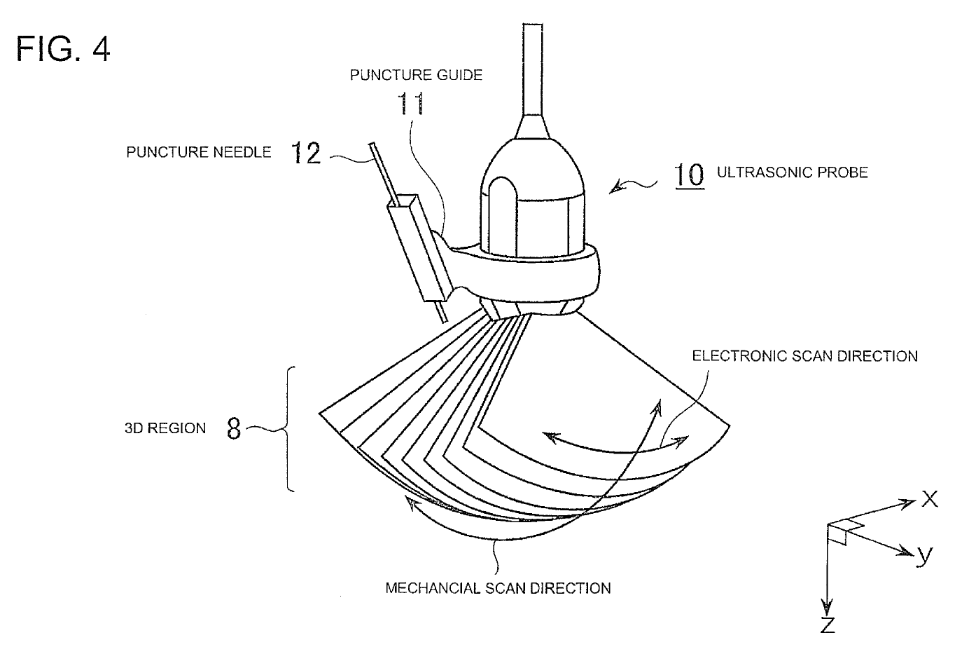

FIG. 4 is an explanatory view which shows scans in the electronic scan direction and the mechanical scan direction orthogonal to the electronic scan direction which are performed using the ultrasonic probe 10, and also schematically shows a 3D region 8 inside the subject 2 which is captured during scanning. The ultrasonic probe 10 performs an electronic scan in the electronic scan direction in which the piezoelectric elements of the probe array 17 are arrayed, and captures tomographic image data. Then, the ultrasonic probe 10 moves the probe array 17 toward the mechanical scan direction orthogonal to the electronic scan direction and again performs an electronic scan there to capture tomographic image data and repeats this sequence.

The puncture needle 12 is attached to the puncture guide 11 fixed on the gripper 52. The puncture guide 11 is designed to introduce the puncture needle 12 into a sectional plane along the electronic scan direction, at the home position, which is in the center of the mechanical scan direction.

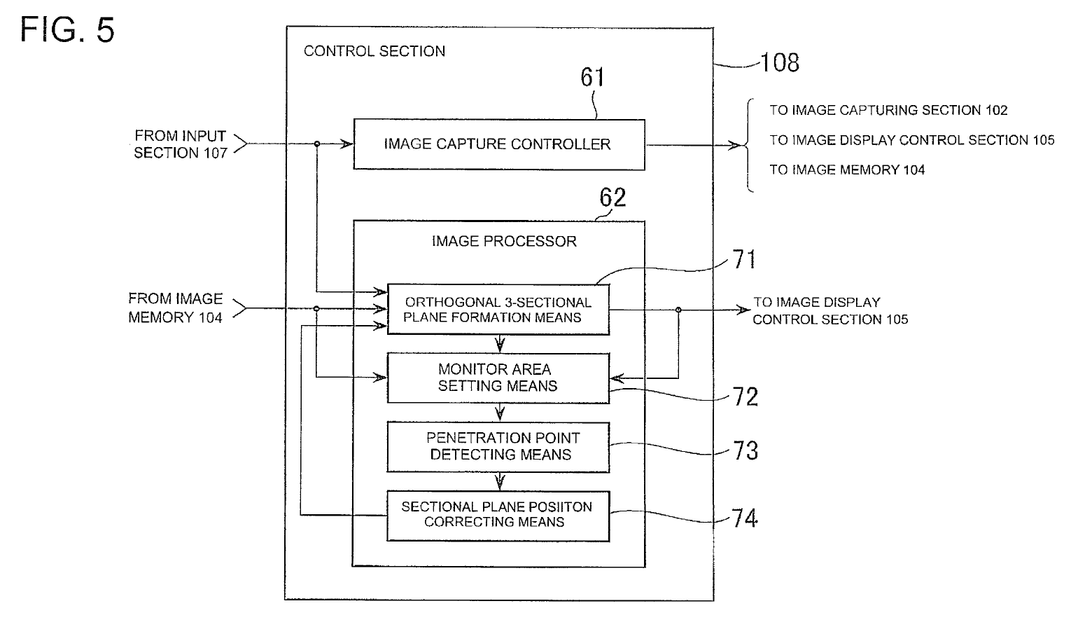

FIG. 5 is a functional block diagram showing the functional structure of the control section 108. The control section 108 includes an image capture controller 61 and an image processor 62 and further the image processor 62 includes an orthogonal 3-sectional plane formation means 71, a monitor area setting means 72, a penetration point detecting means 73 and a sectional plane position correcting means 74.

The image capture controller 61 stores the 3D tomographic image data captured in the 3D region 8 as shown in FIG. 4, in a 3D memory area of the image memory 104. The 3D memory area is a virtual address space that corresponds to the 3D region 8 where the captured tomographic image data in different spots of the 3D region 8 are stored at corresponding addresses.

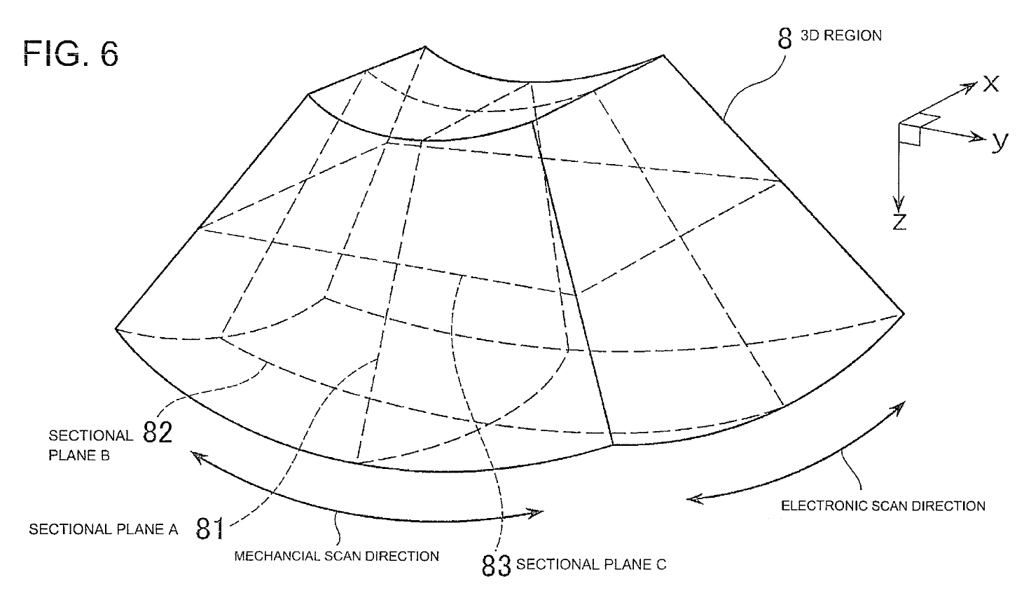

The orthogonal 3-sectional plane formation means 71 forms 2D tomographic image data on three orthogonal sectional planes that are orthogonal to each other in the 3D region 8. FIG. 6 is an explanatory view schematically showing three orthogonal sectional planes set in the 3D region 8. The three orthogonal sectional planes are sectional plane A 81, sectional plane B 82, and sectional plane C 83.

The sectional plane A 81 is parallel to the x-z axis plane and represents a sectional plane in the electronic scan direction at the home position. In default configuration, the sectional plane A 81 is the introduction sectional plane of into which the puncture needle 12 is introduced by the puncture guide 11. The sectional plane B 82 is parallel to the y-z axis plane and represents a sectional plane in the mechanical scan direction. The sectional plane C 83 is parallel to the x-y axis plane and is an opposite sectional plane facing the plane of contact of the ultrasonic probe 10 with the subject 2. The position of the sectional plane A 81 in the mechanical scan direction, the position of the sectional plane B 82 in the electronic scan direction and the depth position of the sectional plane C 83 from the plane of contact of the ultrasonic probe 10 with the subject 2 can be changed by an instruction from the input section 107.

The orthogonal 3-sectional plane formation means 71 extracts tomographic image data from 3D memory areas of the image memory 104 which correspond to the sectional plane A 81, sectional plane B 82 and sectional plane C 83 of the 3D region 8 and displays it on the display section 106. If there is no image data in the 3D memory areas directly corresponding to the sectional plane A 81, sectional plane B 82 and sectional plane C 84, the orthogonal 3-sectional plane formation means 71 generates image data by interpolation, etc and displays it.

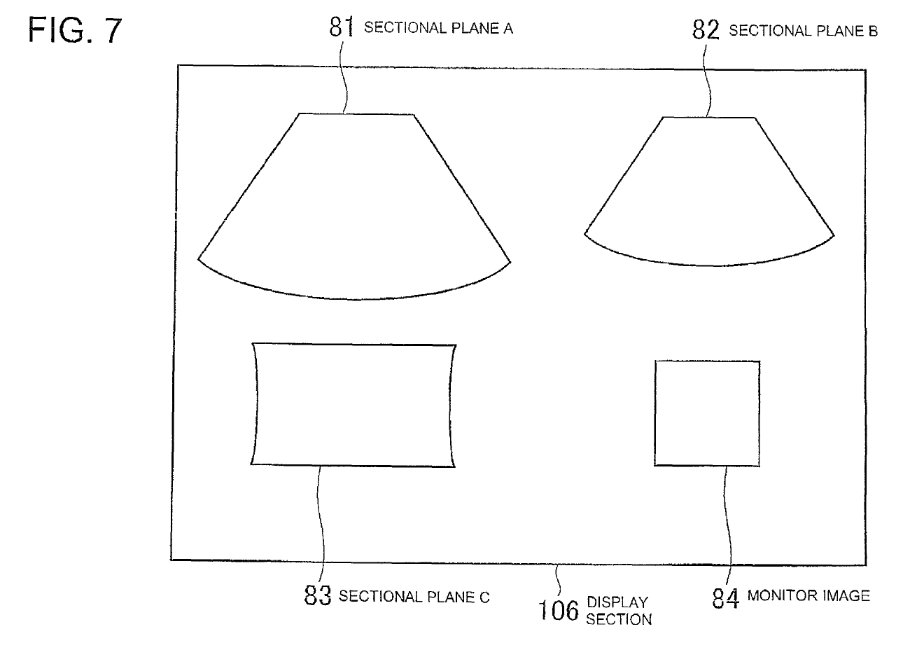

FIG. 7 shows an example of tomographic images of the sectional plane A 81, sectional plane B 82 and sectional plane C 83 that are shown on the display section 106. The sectional plane A 81 is in a left upper part of the screen, the sectional plane B 82 is in a right upper part of it, and the sectional plane C 83 is in a left lower part of it. For these tomographic images, the frame rate at which the images are rewritten is lower than that for a single 2D B mode image but the image data is updated almost in real time. The monitor image 84 shown in a right lower part of the screen in FIG. 7 will be described later.

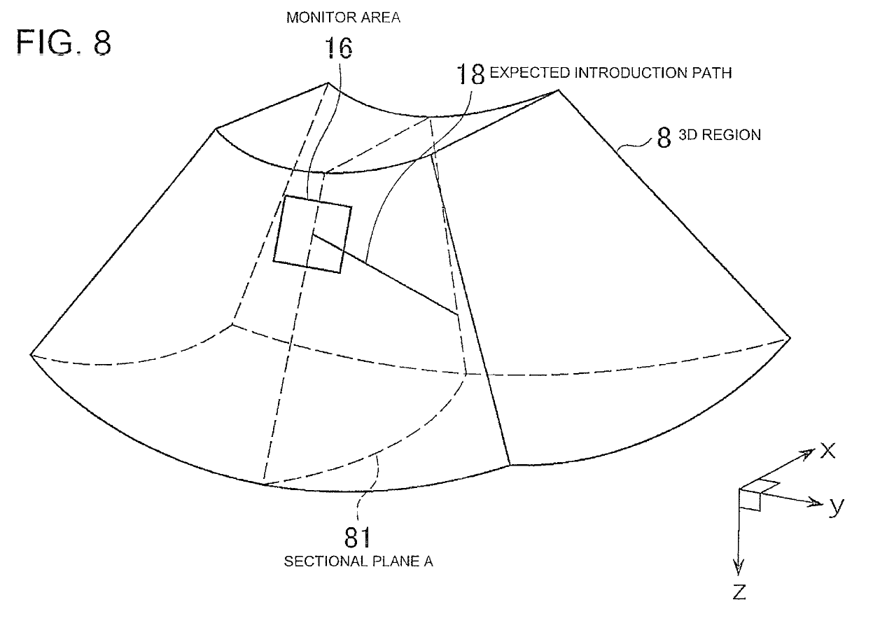

The monitor area setting means 72 sets a monitor memory area in the 3D memory area of the image memory 104 corresponding to the 3D region 8. FIG. 8 is an explanatory view showing the monitor area 16 of the 3D region 8 of the subject 2 corresponding to the monitor memory area of the image memory 104.

FIG. 8 shows the 3D region 8 and the sectional plane A 81, one of the three orthogonal sectional planes. An expected introduction path 18 of the puncture needle 12 along the sectional plane A 81 is indicated in the sectional plane A 81. The expected introduction path 18 is predetermined by the monitor area setting means 72 depending on the shape and mechanical positional relation of the puncture guide 11 attached to the ultrasonic probe 10.

The monitor area setting means 72 sets a monitor area 16 at a spot where the expected introduction path 18 enters the 3D region 8. The monitor area 16 is a rectangular planar area and the center of this planar area is supposed to be a point where the expected introduction path penetrates. This planar area is supposed to be a plane so angled as to be almost orthogonal to the expected introduction path 18; however, the planar area need not necessarily be so angled as to be orthogonal as far as its size is enough to ensure that the actually introduced puncture needle 12 penetrates this planar area. Concretely the monitor area setting means 72 also sets a monitor memory area corresponding to the monitor area 16, in the 3D memory area of the image memory 104.

The monitor area setting means 72 sends 2D tomographic image data on the monitor area 16 to the image display control section 105 and displays it on the display section 106. In this display, the 2D tomographic image data is displayed as a monitor image 84 in a right lower part of the display screen as on the display section 106 shown in FIG. 7.

When an instruction to specify a point in the monitor image 84 is given by a means such as a cursor from the input section 107, the penetration point detecting means 73 obtains data on the position in the 3D region 8 for the specified point in the image. The point specified here is a penetration point at which the puncture needle 12 penetrates the monitor area 16 as will be described later.

The sectional plane position correcting means 74 makes the position of the sectional plane A 81 in the mechanical scan direction coincide with the penetration point of the puncture needle 12 according to positional data on the penetration point of the puncture needle 12 as obtained by the penetration point detecting means 73.

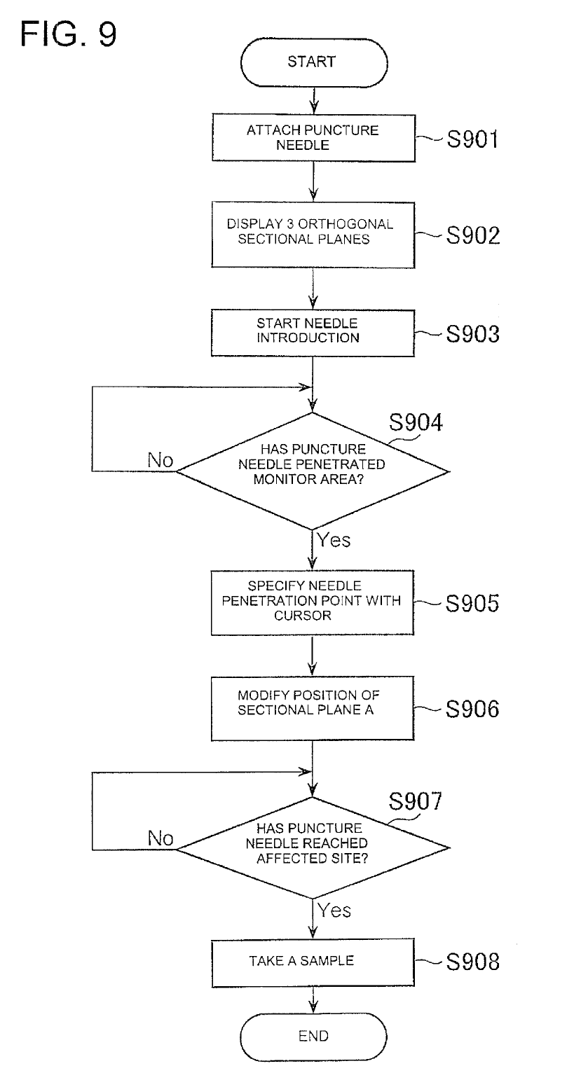

Next, how the image processor 62 operates concretely will be outlined. FIG. 9 is a flowchart showing operation of the image processor 62. First, the operator attaches the puncture guide 11 to the ultrasonic probe 10 and attaches the puncture needle 12 to the puncture guide 11 (Step S901). Then, the operator enters data such as the ultrasonic probe 10's scan mode and the probe array 17's maximum oscillation angle and speed and starts operation of the ultrasonic probe 10 so that three orthogonal sectional planes as shown in FIG. 7 are displayed using the captured 3D tomographic image data (Step S902).

Then, the operator puts the cover 61 of the ultrasonic probe 10 on the subject 2 and starts introduction of the puncture needle 12 (Step S903). The operator introduces the needle referring to the three orthogonal sectional plane images displayed on the display section 106; first the operator refers to the monitor image 84 and decides whether the puncture needle 12 has penetrated the monitor area 16 (Step S904).

Since the puncture needle 12 is a needle with an outside diameter of approximately 1.0 mm, its image appearing in the monitor area 16 almost orthogonal to the introduction direction should be small. However, the cutting edge of the puncture needle 12 is structured as shown in FIG. 2 and the reflected ultrasonic echo from the cutting edge has a higher intensity than that from the tissue and thus when the cutting edge of the puncture needle 12 penetrates the monitor area 16, it is represented as a high-brightness spot in the monitor image 84. The operator can confirm the puncture needle 12's penetration and its penetration point by the presence of the high brightness spot in the monitor image 84.

If there is no high-brightness spot in the monitor image 84, the operator determines that the puncture needle 12 has not penetrated the monitor area 16 (“No” at Step S904) and continues watching the monitor image 84. If there is a high-brightness spot in the monitor image 84, the operator determines that the puncture needle 12 has penetrated the monitor area 16 (“Yes” at Step S904) and specifies the high-brightness spot as the penetration point of the puncture needle 12 using a cursor or the like at the input section 107 (Step S905).

Then, the operator corrects the position of the sectional plane A 81 as the introduction sectional plane in the mechanical scan direction according to the penetration point data indicating the specified penetration point of the puncture needle 12 (Step S906) so that the sectional plane A 81 includes the high-brightness spot, namely the penetration point of the puncture needle 12. Here, the penetration point detecting means 73 identifies the specified high-brightness spot as the penetration point.

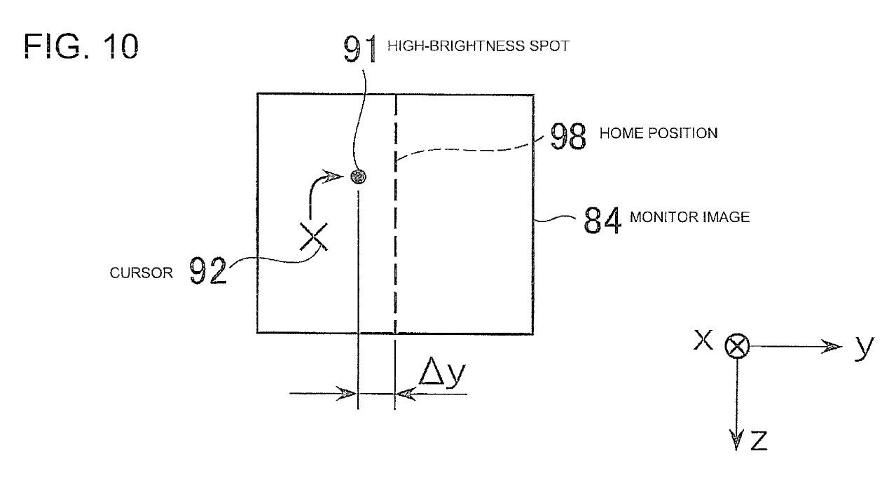

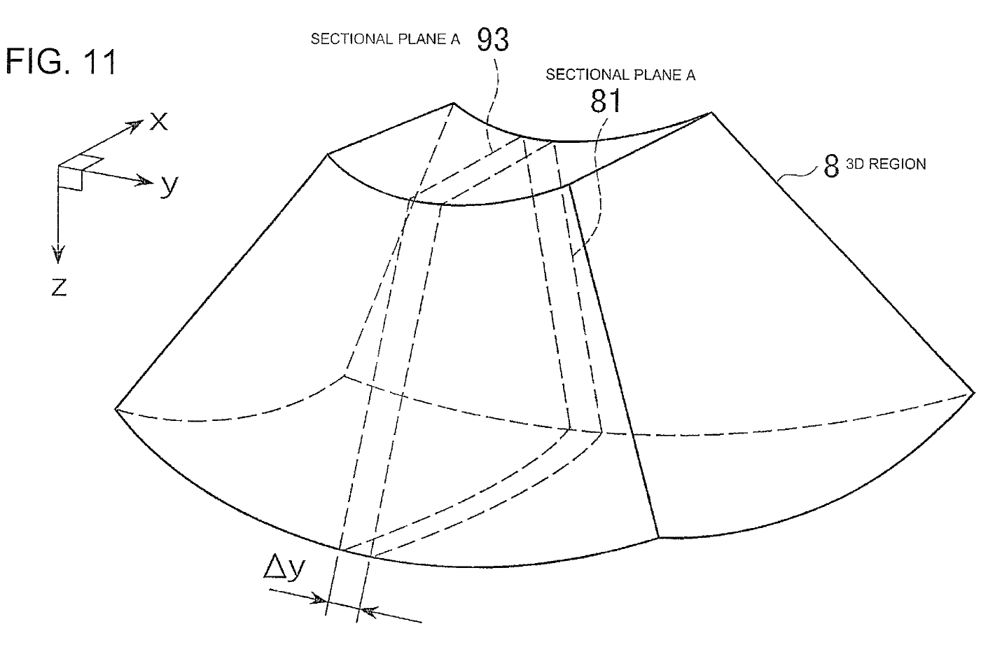

FIG. 10 shows an example of the monitor image 84 in which a high-brightness spot 91 is seen. FIG. 11 is an explanatory view showing a correction of the position of the sectional plane A 81. The monitor image 84 includes the high-brightness spot 91 and the operator moves the cursor 92, an X mark, to the high-brightness spot 91 and specifies the point at the input section 107. For example, the penetration point detecting means 73 calculates moved distance Δy from the home position 98 in the center of the monitor image 84 as indicated by broken line in the y-axis direction. Then, the sectional plane position correcting means 74 moves the sectional plane A 81 at the home position 98 by Δy in the y-axis direction and displays a new sectional plane A 93 on the display section 106 as the introduction sectional plane.

Then, the operator continues introduction work while watching the three orthogonal sectional planes on the display section 106 and decides whether the puncture needle 12 has reached the affected site (Step S907), and if it has not reached the affected site (“No” at Step S907), the operator continues introduction work or if it has reached the affected site (“Yes” at Step S907) and takes a sample (Step S908) and ends the process.

As mentioned above, in the first embodiment, the monitor area 16 is set for the expected introduction path 18 of the puncture needle 12 and the penetration point of the puncture needle 12 which has penetrated the monitor area 16 is detected and according to this penetration point data, the position of the sectional plane A 81 in the mechanical scan direction is corrected; therefore, the actual introduction path of the puncture needle 12 is properly represented in the sectional plane A as the introduction sectional plane.

Also, although in the first embodiment the penetration point detecting means 73 detects the position of the high-brightness spot 91 by a manual procedure based on an instruction given by the operator at the input section 107, the intensity of reflected ultrasonic echo from the cutting edge of the puncture needle 12 is high enough and thus it is also possible to provide an automatic detecting means to detect the penetration point of the puncture needle 12 automatically by specifying a threshold and detecting the position of a pixel in the monitor image 84 which has a brightness value beyond the threshold. Furthermore, the automatic detecting means can find the high-brightness pixel position where the puncture needle surely penetrates, using a plurality of temporally successive monitor images 84 including the time when the cutting edge of the puncture needle 12 penetrates the monitor area 16 in the process of introduction. The sectional plane position correcting means 74 can automatically correct the position of the sectional plane A according to automatically detected penetration point data.

Furthermore, the penetration point detecting means 73, as an automatic detecting means, can also use a plurality of temporally successive monitor images 84 to detect the point of penetration of the monitor area 16 by the puncture needle according to brightness change, based on differences among these monitor images 84 captured at different times. In this case, the position of the pixel where brightness change occurs is identified as the penetration point of the puncture needle.

Also, although in the first embodiment the ultrasonic probe 10 mechanically scans the probe array 17, a 2D probe array that has piezoelectric elements two-dimensionally arranged in an array pattern may be used instead. In this case, 3D tomographic image data can be captured only by an electronic scan.

Although in the first embodiment the monitor area setting means 72 sets a monitor area 16 along the expected introduction path 18 to monitor the introduction path of the puncture needle 12, it is also possible to set two or more monitor areas along the expected introduction path to monitor the actual introduction path of the puncture needle 12 more accurately and correct the sectional plane position of the sectional plane A properly.

The structure of an ultrasonic imaging apparatus according to the second embodiment is exactly the same as that of the ultrasonic imaging apparatus 100 as shown in FIGS. 1 to 5 and detailed description of the structure is omitted.

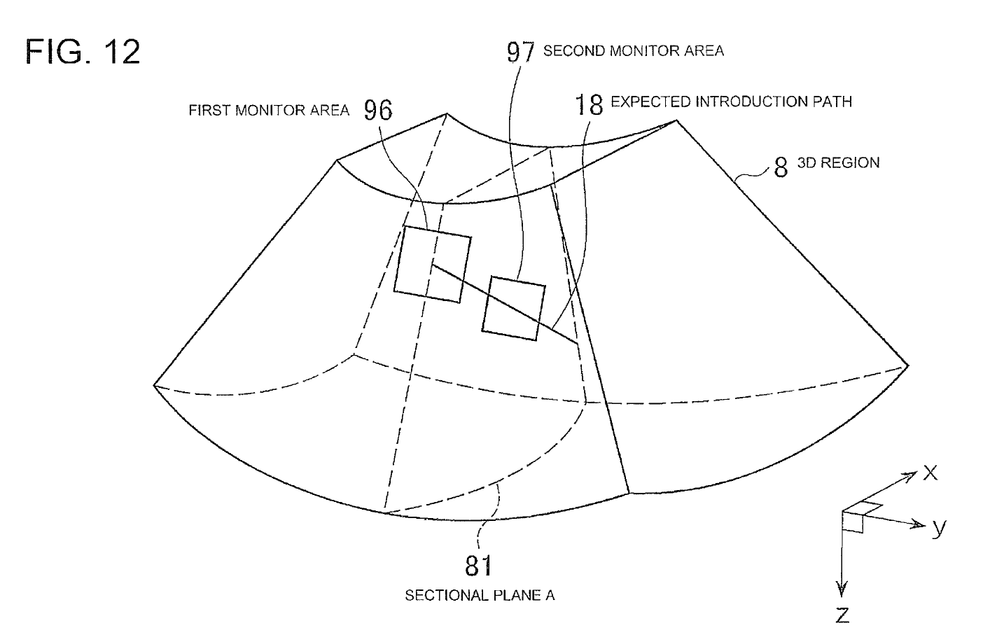

FIG. 12 is an explanatory view of the second embodiment which corresponds to FIG. 8, showing an example that two monitor areas, a first monitor area 96 and a second monitor area 97, are set along the expected introduction path 18 in the 3D region 8. Like the monitor area 16, the first monitor area 96 is located in the periphery where the puncture needle 12 begins to be introduced into the 3D region 8 and the second monitor area 97 is located in the center of the 3D region 8.

The monitor area setting means 72 sets a first monitor memory area for the first monitor area 96 and a second memory area for the second monitor area 97 in the 3D memory area of the image memory 104.

Using 2D tomographic image data in the first monitor memory area and second monitor memory area, the penetration point detecting means 73 detects the penetration points where the puncture needle 12 penetrates the first monitor area 96 and the second monitor area 97, exactly in the same way as for the monitor area 16.

The sectional plane position correcting means 74 corrects the sectional plane position of the sectional plane A 81 as the introduction sectional plane according to the data on the two penetration points. This sectional plane is not always orthogonal to the sectional plane B 82 and the sectional plane C 83 and therefore image data on this sectional plane is formed from 3D tomographic image data by interpolation or the like as appropriate so as to include the detected two penetration points of the puncture needle 12.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus comprising:
an ultrasonic probe configured to capture 3D tomographic image data of a subject;
a puncture guide sized to be held by said ultrasonic probe;
a puncture needle coupled to said puncture guide and configured to be introduced into a 3D region of the subject;
an image memory configured to store the 3D tomographic image data; and
an image processor configured to form, according to the 3D tomographic image data, image data on an introduction sectional plane including an expected introduction path along which the introduction of said puncture needle into the 3D region is expected to be made, wherein, said image processor comprises:
a monitor area setting device configured to set, in a 3D memory area of the image memory corresponding to the 3D region, a monitor memory area which corresponds to a planar monitor area in the 3D region which the expected introduction path penetrates in the vicinity of the center;
a penetration point detecting device configured to detect a point of penetration of the monitor area by said puncture needle according to tomographic image data in the monitor memory area when said puncture needle penetrates the monitor area; and
a sectional plane position correcting device configured to correct the position of the introduction sectional plane to include the penetration point.

2. The ultrasonic imaging apparatus of claim 1, wherein said ultrasonic probe comprises:
a probe array comprising a plurality of piezoelectric elements that are one-dimensionally arranged in one of an arc pattern a linear pattern; and
a mechanical scan device configured to mechanically scan said probe array repeatedly in a mechanical scan direction orthogonal to a direction of the one-dimensional arrangement of said probe array.

3. The ultrasonic imaging apparatus of claim 2, wherein said puncture guide is configured to introduce said puncture needle into a sectional plane including the direction of the arrangement of said probe array, wherein the sectional plane is located in the center of a scan area to be mechanically scanned.

4. The ultrasonic imaging apparatus of claim 3, wherein said image processor is configured to identify the sectional plane as the introduction sectional plane.

5. The ultrasonic imaging apparatus of claim 1, wherein said ultrasonic probe comprises a 2D probe array comprising a plurality of piezoelectric elements that are two-dimensionally arranged in a planar pattern.

6. The ultrasonic imaging apparatus of claim 1, wherein said image processor further comprises an orthogonal 3-sectional plane formation device configured to form tomographic image data on three orthogonal sectional planes in the 3D region, wherein the three orthogonal sectional planes include the introduction sectional plane.

7. The ultrasonic imaging apparatus of claim 6, further comprising a display section configured to display tomographic image data within the three orthogonal sectional planes.

8. The ultrasonic imaging apparatus of claim 7, wherein said monitor area setting device is configured to extract tomographic image data in the monitor memory area from the image memory and to display tomographic images of the tomographic image data on said display section.

9. The ultrasonic imaging apparatus of claim 8, wherein said penetration point detecting device comprises a cursor position detecting device configured to detect a cursor for specifying a point in the displayed tomographic image and the position of the cursor in the tomographic image and to identify the specified point as the penetration point.

10. The ultrasonic imaging apparatus of claim 1, wherein said penetration point detecting device comprises an automatic detecting device configured to extract tomographic image data in the monitor memory area from the 3D tomographic image data and to automatically detect the penetration point of said puncture needle as included in a tomographic image of the tomographic image data.

11. The ultrasonic imaging apparatus of claim 10, wherein said automatic detecting device is configured to automatically detect the pixel position of a high-brightness spot beyond a threshold in the tomographic image, as the penetration point.

12. The ultrasonic imaging apparatus of claim 10, wherein when the 3D tomographic image data captured temporally successively is stored in said image memory, said automatic detecting device is configured to:
extract temporally successive tomographic image data in the monitor memory area; and
automatically detect the penetration point from tomographic images of the extracted tomographic image data.

13. The ultrasonic imaging apparatus of claim 10, wherein when the 3D tomographic image data captured temporally successively is stored in said image memory, the automatic detecting device is configured to:
extract temporally successive 2D tomographic image data in the monitor memory area;
differentiate between two 2D tomographic images captured at different times in 2D tomographic images of the plural 2D tomographic image data; and
automatically detect the pixel position of a spot beyond a threshold as included in a difference tomographic image obtained by the differentiation, as the penetration point.

14. The ultrasonic imaging apparatus of claim 1, wherein said monitor area setting device comprises a first monitor memory area in said image memory corresponding to a first monitor area located in the 3D region's periphery where said puncture needle begins to be introduced into the 3D region.

15. The ultrasonic imaging apparatus of claim 14, wherein said monitor area setting device comprises a second monitor memory area in said image memory corresponding to a second monitor area located within the 3D region.

16. The ultrasonic imaging apparatus of claim 15, wherein, according to the tomographic image data in the first monitor memory area and the second monitor memory area, said penetration point detecting device is configured to detect a first penetration point and a second penetration point where said puncture needle penetrates the first monitor area and the second monitor area respectively.

17. The ultrasonic imaging apparatus of claim 16, wherein said sectional plane position correcting device is configured to correct the position of the introduction sectional plane so as to include the first penetration point and the second penetration point.

18. A method for ultrasonic imaging, said method comprising:
capturing 3D tomographic image data of a subject;
introducing a puncture needle into a 3D region of the subject using a puncture guide;
storing the captured 3D tomographic image data;
forming image data on an introduction sectional plane that includes an expected introduction path along which the introduction of the puncture needle into the 3D region is expected to be made;
setting a monitor memory area that corresponds to a planar monitor area in the 3D region, wherein the expected introduction path passes through the 3D region in a vicinity of a center;
detecting a point of penetration of the monitor area by the puncture needle according to tomographic image data in the monitor memory area when the puncture needle penetrates the monitor area; and
correcting the position of the introduction sectional plane to include the penetration point for display to an operator.

19. The method of claim 18, further comprising mechanically scanning a probe array that includes a plurality of piezoelectric elements arranged in one of a one-dimensional pattern and a two-dimensional patter.

20. An ultrasonic imaging apparatus comprising:
an ultrasonic probe configured to capture 3D tomographic image data of a subject;
a puncture guide sized to be held by said ultrasonic probe;
a puncture needle coupled to said puncture guide and configured to be introduced into a 3D region of the subject;
an image memory configured to store the 3D tomographic image data;
an image processor configured to form, according to the 3D tomographic image data, image data on an introduction sectional plane including an expected introduction path along which the introduction of said puncture needle into the 3D region is expected to be made, wherein, said image processor comprises:
a monitor area setting device configured to set, in a 3D memory area of the image memory corresponding to the 3D region, a monitor memory area which corresponds to a planar monitor area in the 3D region which the expected introduction path penetrates in the vicinity of the center;
a penetration point detecting device configured to detect a point of penetration of the monitor area by said puncture needle according to tomographic image data in the monitor memory area when said puncture needle penetrates the monitor area; and
a sectional plane position correcting device configured to correct the position of the introduction sectional plane to include the penetration point; and
a display section configured to display the tomographic image data to an operator.
